(19) United States
(12) Patent Application Publication
(10) Pub. No.: US 2022/0241264 A1
(43) Pub. Date: Aug. 4, 2022

(54) METHODS FOR PREVENTION AND TREATMENT OF FIBROMYALGIA BY CONTACT VASODILATORS

(71) Applicant: Assa Weinberg, Los Angeles, CA (US)

(72) Inventor: Assa Weinberg, Los Angeles, CA (US)

(21) Appl. No.: 17/721,742

(22) Filed: Apr. 15, 2022

Related U.S. Application Data
(63) Continuation of application No. PCT/US2020/046628, filed on Aug. 17, 2020.
(60) Provisional application No. 62/916,964, filed on Oct. 18, 2019.

(51) Int. Cl. A61K 31/4439; A61K 47/24; A61K 31/4422; A61P 25/02

(52) U.S. Cl. CPC A61K 31/4439; A61P 25/02; A61K 31/4422; A61K 47/24

(57) ABSTRACT

Methods are provided to prevent and to treat fibromyalgia by using a vasodilator such as an angiotensin receptor blocker, ACE inhibitor, calcium channel blocker, phosphodiesterase inhibitor, alpha blocker, or beta blocker. More particularly, the methods do not employ orally administered vasodilators, but instead vasodilators that are administered through contact with the epidermis, e.g., in topical or other form suitable for contact with tissues to be treated. The compositions promote neo capillary formation, whereby symptoms of fibromyalgia are treated, reduced, or ameliorated.

Any and all priority claims identified in the Application Data Sheet, or any correction hereto, are hereby incorporated by reference under 37 CFR 1.57. This application is a continuation of and claims benefit of Application No. PCT/US2020/046628, filed Aug. 17, 2020, which claims benefit of U.S. Provisional Application No. 62/916,964, filed Oct. 18, 2019. The aforementioned application is incorporated by reference herein in its entirety, and is hereby expressly made a part of this application.

### FIELD OF THE INVENTION

Methods are provided to prevent and to treat fibromyalgia by using a vasodilator such as an angiotensin receptor blocker, ACE inhibitor, calcium channel blocker, phosphodiesterase inhibitor, alpha blocker, or beta blocker. More particularly, the methods do not employ orally administered vasodilators, but instead vasodilators that are administered through contact with the epidermis, e.g., in topical or other form suitable for contact with tissues to be treated. The compositions promote neo capillary formation, whereby symptoms of fibromyalgia are treated, reduced, or ameliorated.

### BACKGROUND OF THE INVENTION

Fibromyalgia is the world's most common syndrome of chronic musculoskeletal pain. Chronic fatigue and sleep disordered symptoms were added to the total required diagnostic criteria for fibromyalgia by the American College of Rheumatology (ACR). The syndrome is defined purely by symptomatic criteria, which has made its presence controversial. Fibromyalgia syndrome was first described in 1990 by the American college of Rheumatology. Since than the criteria have been revised four times. The latest revision of 2016 includes the following three criteria: the presence of pain in four out of five regions of the multisite pain regions; the presence of chronic fatigue and chronic sleep disorder; and pain, fatigue, and sleep disturbances present for no less than three months.

The prevalence of this syndrome in the US population and in other countries is 2% of the total population (3.4% for women and 0.5% for men). Fibromyalgia can be present in children and adults. It is the most common cause of generalized diffuse musculoskeletal pain in women aged 20 to 55. The prevalence of fibromyalgia is age related. The prevalence increases with age and is nearly doubled by the age of 60. The core symptom of fibromyalgia is widespread chronic musculoskeletal pain or multisite pain. Fatigue and sleep disorders are cardinal criteria in fibromyalgia.

Cognitive disturbances, or “fibro fog”, associated with fibromyalgia are different from those found in psychiatric syndromes. Subjective cognitive deficits are common, and are believed to be associated with the presence of chronic pain and depression. Depression and anxiety are present in 30 to 50% of patients at the time of diagnosis of fibromyalgia—a frequency three times that of the general population. Headache is present in 50% of fibromyalgia patients, and includes migraines and tension headaches. The most common symptom observed in physical examination is the presence of tender points (as described in the 1990 ACR diagnostic criteria). However, the search for and presence of tender points was discarded in successive revisions of the criteria in 2010, 2011, and 2016 by the ACR and by the Analgesic, Anesthetic, and Addiction Clinical Trial Translations, Innovations, Opportunities, and Networks (ACTION), Food and Drug Administration (FDA), the American Pain Society, and pain taxonomy in 2013.

Fibromyalgia is a chronic pain syndrome with unknown etiology. In the last 20 years a major scientific effort to unravel the cause of fibromyalgia and to generate treatment options was undertaken. More than five thousand articles were published in the scientific literatures on the physiopathology of fibromyalgia and more than six thousand articles were published on the treatment of fibromyalgia according to PUB MED data. These efforts included genetic studies, altered pain processing studies, pain summation studies, defective pain inhibition studies, studies of pain receptors and pain neuropeptides studies, neuroimaging studies, functional MRI studies, positron emission tomography studies, studies of cognitive and affective factors, morphometric brain studies, positron magnetic resonance spectrometry studies, systematic reviews of imaging studies, sleep studies, neurohormonal studies (e.g., of corticotrophin releasing factors, cortisol, growth hormone, autonomic nervous system, immune system in fibromyalgia autoantibodies in fibromyalgia, and cytokines), and peripheral pain mechanism studies (e.g., of trigger points, small fiber neuropathy, muscle metabolic alteration ATP and phosphocreatinine, muscle fiber and capillaries).

Current methods of treatment of fibromyalgia center on education, physiotherapy, and drugs. Studies of the impact of education and exercises on fibromyalgia revealed significantly fewer diagnostic tests conducted and fewer referrals after the fibromyalgia diagnosis. A reduction in imaging studies, primary care visits and fewer drugs prescription than prior to diagnosis was also observed. A decline in self reporting pain, stiffness, anxiety and depression was observed one month after 1.5 days of education. Low resistance exercise, aquatic exercise, and aerobic exercise reduce pain and stiffness in fibromyalgia. Six medications, including antidepressants and anticonvulsants, have been intensively studied in fibromyalgia. Each of the drugs so tested showed efficacy against placebo in randomized trials and meta analysis. Only three of the drugs are FDA approved, including duloxetine, milnacioram, and pregabalin. Other drugs tested include amitriptyline and cyclobenzaprine (both of which are tricyclics) and gabapentin (an anticonvulsant). Despite their superiority over placebo, a 2012 meta analysis of antidepressants used in fibromyalgia found that only a minority of patients gained a substantial result. Only moderate improvement in pain and sleep were achieved by the majority of such patients, and only minimal impact on fatigue and quality of life was observed. Substantial number of patients drop out of the studies because of side effects. Unfortunately, long term follow up studies found that pain, fatigue, sleep disorders, anxiety, and depression were essentially unchanged. Only 25% of patients experience moderate improvement over time. 31% of patients declared that they are disabled and 26.9% received a disability pension.

In spite of the large scientific insight that was acquired over the last 20 years on fibromyalgia, three enigmas remain. The cause is still unknown; it is not biopsy based; and the diagnosis criteria are cumbersome, unusually and uniquely based on the veracity of a patient filled form. Treatment achieves only temporary symptom relief, heretofore, there is no cure in sight.

### SUMMARY OF THE INVENTION

In view of the limitations of the current methods of prevention and treatment of fibromyalgia, a new method of prevention and treatment is desirable.

Herein provided, is an answer to the three enigmas summarized above: a tissue based pathogenesis, new diagnostic criteria, and new treatment.

Provided is a method of applying a pharmaceutical preparation in an effective amount of a vasodilator, directly to the epidermis in the region of affected areas to prevent or treat fibromyalgia. Such a vasodilator can be considered a contact vasodilator. This is in contrast to vasodilators administered in oral form for systemic delivery, e.g., in the treatment of conditions such as high blood pressure. Vasodilators administered in oral form for systemic delivery for treatment of conditions such as high blood pressure do not exhibit an impact on fibromyalgia. It has been surprisingly learned that formulation of the vasodilator into a form for contact with an epidermis to be treated results in lasting physiological changes, e.g., structural changes to the epidermis or adjacent tissue that can last, e.g., for several months or longer, providing relief to the symptoms of fibromyalgia.

Accordingly, in a generally applicable first aspect (i.e., independently combinable with any of the aspects or embodiments identified herein), a pharmaceutical composition for the treatment or prophylaxis of fibromyalgia is provided, comprising: at least one vasodilator; and at least one pharmaceutical excipient.

In a generally applicable embodiment (i.e., independently combinable with any of the aspects or embodiments identified herein) of the first aspect, the vasodilator is a contact vasodilator.

In a generally applicable embodiment (i.e., independently combinable with any of the aspects or embodiments identified herein) of the first aspect, the contact vasodilator is a calcium channel blocker.

In a generally applicable embodiment (i.e., independently combinable with any of the aspects or embodiments identified herein) of the first aspect, the at least one calcium channel blocker is a dihydropyridine selected from the group consisting of nifedipine, isradipine, felodipine, amlodipine, nicardipine, and clevidipine.

In a generally applicable embodiment (i.e., independently combinable with any of the aspects or embodiments identified herein) of the first aspect, the at least one calcium channel blocker is a non dihydropyridine selected from the group consisting of verapamil and diltiazem.

In a generally applicable embodiment (i.e., independently combinable with any of the aspects or embodiments identified herein) of the first aspect, the contact vasodilator is an ACE inhibitor.

In a generally applicable embodiment (i.e., independently combinable with any of the aspects or embodiments identified herein) of the first aspect, the ACE inhibitor is selected from the group consisting of benazepril, captopril, enalapril, fosinopril, lisinopril, moexipril, perindopril, quinapril, ramipril, and trandolapril.

In a generally applicable embodiment (i.e., independently combinable with any of the aspects or embodiments identified herein) of the first aspect, the contact vasodilator is an angiotensin receptor blocker.

In a generally applicable embodiment (i.e., independently combinable with any of the aspects or embodiments identified herein) of the first aspect, the angiotensin receptor blocker is selected from the group consisting of azilsartan, candesartan, eprosartan, irbesartan, losartan, olmesartan, telmisartan, and valsartan.

In a generally applicable embodiment (i.e., independently combinable with any of the aspects or embodiments identified herein) of the first aspect, the pharmaceutical composition is in a form is selected from the group consisting of a cream, a lotion, an ointment, a gel base, a foam, a powder, an aerosol spray, an oil, a liquid, and a suspension.

In a generally applicable embodiment (i.e., independently combinable with any of the aspects or embodiments identified herein) of the first aspect, the pharmaceutical composition is formulated as a liquid or a suspension of the at least one vasodilator.

Accordingly, in a generally applicable second aspect (i.e., independently combinable with any of the aspects or embodiments identified herein), a method is provided for the treatment or prophylaxis of a symptom of fibromyalgia in a patient in need thereof, comprising: administering an effective amount of the pharmaceutical composition according to the first aspect or any of its embodiments, whereby a symptom of fibromyalgia in patients is treated or prevented.

Accordingly, in a generally applicable first aspect (i.e., independently combinable with any of the aspects or embodiments identified herein), a pharmaceutical composition for the treatment or prophylaxis of fibromyalgia syndrome is provided, comprising: at least one vasodilator; and at least one pharmaceutical excipient.

In a generally applicable embodiment (i.e., independently combinable with any of the aspects or embodiments identified herein) of the first aspect, the pharmaceutical composition is in a form adapted for topical administration to an epidermis.

In a generally applicable embodiment (i.e., independently combinable with any of the aspects or embodiments identified herein) of the first aspect, the pharmaceutical composition is in a form selected from the group consisting of a cream, a lotion, an ointment, a gel base, a foam, a powder, an aerosol spray, an oil, a liquid, and a suspension.

In a generally applicable embodiment (i.e., independently combinable with any of the aspects or embodiments identified herein) of the first aspect, the vasodilator is a calcium channel blocker.

In a generally applicable embodiment (i.e., independently combinable with any of the aspects or embodiments identified herein) of the first aspect, the at least one calcium channel blocker is a dihydropyridine selected from the group consisting of nifedipine, isradipine, felodipine, amlodipine, nicardipine, and clevidipine.

In a generally applicable embodiment (i.e., independently combinable with any of the aspects or embodiments identified herein) of the first aspect, the at least one calcium channel blocker is a non dihydropyridine selected from the group consisting of verapamil and diltiazem.

In a generally applicable embodiment (i.e., independently combinable with any of the aspects or embodiments identified herein) of the first aspect, the vasodilator is an ACE inhibitor.

In a generally applicable embodiment (i.e., independently combinable with any of the aspects or embodiments identified herein) of the first aspect, the ACE inhibitor is selected from the group consisting of benazepril, captopril, enalapril, fosinopril, lisinopril, moexipril, perindopril, quinapril, ramipril, and trandolapril.

In a generally applicable embodiment (i.e., independently combinable with any of the aspects or embodiments identified herein) of the first aspect, the vasodilator is an angiotensin receptor blocker.

In a generally applicable embodiment (i.e., independently combinable with any of the aspects or embodiments identified herein) of the first aspect, the angiotensin receptor blocker is selected from the group consisting of azilsartan, candesartan, eprosartan, irbesartan, losartan, olmesartan, telmisartan, and valsartan.

In a generally applicable embodiment (i.e., independently combinable with any of the aspects or embodiments identified herein) of the first aspect, the vasodilator is a nitrate.

In a generally applicable embodiment (i.e., independently combinable with any of the aspects or embodiments identified herein) of the first aspect, the nitrate is selected from the group consisting of nitroglycerin, isosorbide mononitrate and isosorbide dinitrate.

In a generally applicable embodiment (i.e., independently combinable with any of the aspects or embodiments identified herein) of the first aspect, the vasodilator is an alpha blocker.

In a generally applicable embodiment (i.e., independently combinable with any of the aspects or embodiments identified herein) of the first aspect, the alpha blocker is selected from the group consisting of doxazosin, prazosin, and terazosin.

In a generally applicable embodiment (i.e., independently combinable with any of the aspects or embodiments identified herein) of the first aspect, the vasodilator is a beta blocker.

In a generally applicable embodiment (i.e., independently combinable with any of the aspects or embodiments identified herein) of the first aspect, the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

In a generally applicable embodiment (i.e., independently combinable with any of the aspects or embodiments identified herein) of the first aspect, the vasodilator is hydralazine.

In a generally applicable embodiment (i.e., independently combinable with any of the aspects or embodiments identified herein) of the first aspect, the vasodilator is an angiotensin receptor-neprilysin inhibitor.

In a generally applicable embodiment (i.e., independently combinable with any of the aspects or embodiments identified herein) of the first aspect, the angiotensin receptor-neprilysin inhibitor is sacubitril/valsartan.

In a generally applicable second aspect (i.e., independently combinable with any of the aspects or embodiments identified herein), a method is provided for the treatment or prophylaxis of fibromyalgia in a patient in need thereof, comprising: administering an effective amount of the pharmaceutical composition according to any embodiment of the third aspect to a patient in need thereof, whereby a symptom of fibromyalgia, is treated or prevented.

In a generally applicable embodiment (i.e., independently combinable with any of the aspects or embodiments identified herein) of the second aspect, the symptom is pain.

In a generally applicable embodiment (i.e., independently combinable with any of the aspects or embodiments identified herein) of the second aspect, t the symptom is myofacial pain.

In a generally applicable embodiment (i.e., independently combinable with any of the aspects or embodiments identified herein) of the second aspect, the symptom is soft tissue pain.

In a generally applicable third aspect (i.e., independently combinable with any of the aspects or embodiments identified herein), a pharmaceutical composition is provided for the promotion of neo capillary formation, comprising: at least one vasodilator; and at least one pharmaceutical excipient.

In a generally applicable embodiment (i.e., independently combinable with any of the aspects or embodiments identified herein) of the third aspect, the pharmaceutical composition is in a form adapted for topical administration to an epidermis.

In a generally applicable embodiment (i.e., independently combinable with any of the aspects or embodiments identified herein) of the third aspect, the form is selected from the group consisting of a cream, a lotion, an ointment, a gel base, a foam, a powder, an aerosol spray, an oil, a liquid, and a suspension.

In a generally applicable embodiment (i.e., independently combinable with any of the aspects or embodiments identified herein) of the third aspect, the vasodilator is a calcium channel blocker.

In a generally applicable embodiment (i.e., independently combinable with any of the aspects or embodiments identified herein) of the third aspect, the at least one calcium channel blocker is a dihydropyridine selected from the group consisting of nifedipine, isradipine, felodipine, amlodipine, nicardipine, and clevidipine.

In a generally applicable embodiment (i.e., independently combinable with any of the aspects or embodiments identified herein) of the third aspect, the at least one calcium channel blocker is a non dihydropyridine selected from the group consisting of verapamil and diltiazem.

In a generally applicable embodiment (i.e., independently combinable with any of the aspects or embodiments identified herein) of the third aspect, the vasodilator is an ACE inhibitor.

In a generally applicable embodiment (i.e., independently combinable with any of the aspects or embodiments identified herein) of the third aspect, the ACE inhibitor is selected from the group consisting of benazepril, captopril, enalapril, fosinopril, lisinopril, moexipril, perindopril, quinapril, ramipril, and trandolapril.

In a generally applicable embodiment (i.e., independently combinable with any of the aspects or embodiments identified herein) of the third aspect, the vasodilator is an angiotensin receptor blocker.

In a generally applicable embodiment (i.e., independently combinable with any of the aspects or embodiments identified herein) of the third aspect, the angiotensin receptor blocker is selected from the group consisting of azilsartan, candesartan, eprosartan, irbesartan, losartan, olmesartan, telmisartan, and valsartan.

In a generally applicable embodiment (i.e., independently combinable with any of the aspects or embodiments identified herein) of the third aspect, the vasodilator is a nitrate.

In a generally applicable embodiment (i.e., independently combinable with any of the aspects or embodiments identified herein) of the third aspect, the nitrate is selected from the group consisting of nitroglycerin, isosorbide mononitrate and isosorbide dinitrate.

In a generally applicable embodiment (i.e., independently combinable with any of the aspects or embodiments identified herein) of the third aspect, the vasodilator is an alpha blocker.

In a generally applicable embodiment (i.e., independently combinable with any of the aspects or embodiments identified herein) of the third aspect, the alpha blocker is selected from the group consisting of doxazosin, prazosin, and terazosin.

In a generally applicable embodiment (i.e., independently combinable with any of the aspects or embodiments identified herein) of the third aspect, the vasodilator is a beta blocker.

In a generally applicable embodiment (i.e., independently combinable with any of the aspects or embodiments identified herein) of the third aspect, the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

In a generally applicable embodiment (i.e., independently combinable with any of the aspects or embodiments identified herein) of the third aspect, the vasodilator is hydralazine.

In a generally applicable embodiment (i.e., independently combinable with any of the aspects or embodiments identified herein) of the third aspect, the vasodilator is an angiotensin receptor-neprilysin inhibitor.

In a generally applicable embodiment (i.e., independently combinable with any of the aspects or embodiments identified herein) of the third aspect, the angiotensin receptor-neprilysin inhibitor is sacubitril/valsartan.

In a generally applicable fourth aspect (i.e., independently combinable with any of the aspects or embodiments identified herein), a method is provided for the promotion of neo capillary formation in a patient in need thereof, comprising: administering an effective amount of the pharmaceutical composition according to any embodiment of the third aspect to a patient in need thereof, whereby neo capillaries are formed.

Any of the features of an embodiment of the first and second aspects is applicable to all aspects and embodiments identified herein. Moreover, any of the features of an embodiment of the first and second aspects is independently combinable, partly or wholly with other embodiments described herein in any way, e.g., one, two, or three or more embodiments may be combinable in whole or in part. Further, any of the features of an embodiment of the first and second aspects may be made optional to other aspects or embodiments.

### DETAILED DESCRIPTION

A method is provided of applying a pharmaceutical preparation in an effective amount of one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), directly to the epidermis of affected regions to treat, prevent, or ameliorate a symptom of fibromyalgia.

The compositions and methods of the embodiments are useful in the treatment, amelioration, or control of capillary pathogenesis and associated conditions. The compositions and methods of the embodiments are useful for promoting neo capillaries formation, as in the treatment of conditions such as fibromyalgia. Fibromyalgia is a centuries-old enigma. A review of the extensive technical literature points to two prevailing theories, both based on reproducible facts, and both contradicting each other. The diagnosis relies on the veracity of a patient-filled form, unlike the diagnosis of many other conditions. Moreover, the results of current treatment are poor and temporary. Provided herein are compositions and methods founded on a unified hypothesis that reconciles contradictions with respect to the underlying cause(s) of fibromyalgia, namely, the capillary base to fibromyalgia pathogenesis and its long symptoms list and the association with fibromyalgia brain fog and other wide-spread pain (WSP) syndrome. Provided are new diagnostic criteria for fibromyalgia.

The capillary-insufficiency of fibromyalgia permits the formation of new simple physician-dependent diagnostic criteria. These criteria are a proposed modification of the current American College of Rheumatology (ACR) diagnostic criteria. This allows the preservation of the impressive sensitivity and specificity of the ACR criteria. The new criteria require the following:
1) the presence of pain in three bilateral symmetric pain territories for more than three months;
2) the inability to identify the pain areas or affect its intensity by physical exam; and
3) the ability of cold application to increase pain and heat application to reduce pain and morning stiffness.

When patients meet these criteria for at least 3 months, this means that the three components of fibromyalgia fog are already present in most of the patients. Even if none one or two of the components of fibromyalgia fog are present in these patients, the ACR recommended treatment produces the same rate of success and failure of the current ACR criteria. It is easy to see the advantages of the new diagnostic criteria and their relationship to the capillary-insufficiency pathogenesis.

Provided herein is a new treatment for fibromyalgia that has not previously been described. The new treatment consists of direct application of vasodilators to the pain territories. Drugs that produce vasodilation by skin application are now called contact vasodilators. The immediate effect of contact vasodilators is to increasing the blood supply to the pain territories by vasodilation. This increases the oxygen and nutrients delivery to muscle and nerve and reverses the insufficiency state produced by the reduced capillaries density. The outcome is a reduced pain. However, the main effect of these contact vasodilators is to induce the formation of new additional capillaries that correct the fiber/capillaries ratio. Over the last 20 years more than 16 natural endothelial capillaries growth factors have been isolated. They induce and regulate the formations of capillaries and their density in humans. At least four pharmaceutical antibodies that block the activity of these growth factors are now approved in the treatment the capillaries leak that causes macular degeneration. However, no pharmaceutical drug or group of drugs has heretofore been known to induce the formation of new capillaries. Provided herein are methods and compositions that promote neo capillaries formation. This is the main pharmaceutical activity of these contact vasodilators. The discovery of such drugs is of capital importance to patients with fibromyalgia. Contact vasodilators increase the local blood supply to the pain territory. This produces more than a concurrent analgesic effect. The neo capillaries reverse the existent insufficiency which is the base for fibromyalgia pathogenesis. The reversal of the fiber capillary ratio in fibromyalgia opens the possibility for a cure to this disease.

### Fibromyalgia and Capillaries

In spite of the large scientific insight that was acquired over the last 20 years regarding fibromyalgia, three enigmas remain: the cause remains unknown; the diagnosis is cumbersome, based on patients' veracity; and treatment achieves only temporary symptoms relief. Heretofore, no cure was in sight, but provided herein are answers to the three enigmas above: new pathogenesis; new diagnostic criteria; and new treatment.

With respect to a new pathogenesis, fibromyalgia is a vascular syndrome. Specifically, capillary insufficiency syndrome. Many electron microscopy studies support this fact. In many of these studies this fact is even quantitatively present. Figures have been published which demonstrate this, where the electron microscopy findings on muscle of fibromyalgia are compared to normal control. In all such studies, observers noted that the capillary density surrounding each fiber in fibromyalgia muscles was significantly reduced when compared to their density in normal control. A study published in 2012 states that the capillary density was reduced in all patients with fibromyalgia. This relationship between fibers and capillaries is known as the fiber capillary ratio or F/C ratio. This ratio has been studied extensively. Studies in fish and mammals have demonstrated that the F/C ratio exerts a major impact on the development of species.

It is now considered as one of the determining factors in the development of the mammalian species by its cardinal impact on thermo-regulation and homeostasis. In addition to heat regulation, the capillary system is responsible to meet the harsh metabolic demands of the muscular activity. These demands can varies drastically, by a factor of one to ten. By its design the capillary system enables an adequate and variable delivery of oxygen and nutrients to tasks performing muscles It is know well known that insufficient delivery system, known as vascular insufficiency, leads to rapid decline in the muscular system performances and a premature onset of fatigue. The pathological significance of reduced fiber/capillary ratio in humans was recognized only recently. As, unfortunately, the only method that can detect a reduction in capillary density is limited to electron microscopy technology. The electron microscopy observations that documented the reduction of capillary density in fibromyalgia ignored their pathological significance. Hemodynamics consequences of a reduced fiber/capillary ratio are the corner stone of fibromyalgia pathogenesis. The reduced ratio amounts to reduction of oxygen and nutrients delivery. That corresponds to the metabolic demand.

Fibromyalgia capillary system has a visible design flow that prevent it from delivering a sufficient oxygen and nutrients to responds to the metabolic demands as a normal capillary system. The design defect is the reduced fiber/capillary ratio. Studies dating from the nineteenth century revealed the significant hemodynamics consequences of even a small reduction in the tube diameter on fluid flow. This is known as Bernoulli's law or effect. Other studies by electron microscopy confirmed this observations. The studies report that the concentration of ATP molecules are significantly reduced in fibromyalgia muscles ATP molecules are the fuel used by muscle to contract. Without ATP contractions is impossible. Studies over the last 80 years, including one receiving the Nobel price in chemistry in 1997, revealed that the amount of ATP in cells reflect the amount of the blood flow they receive. Their synthesis depends on the delivery of the building components of their structure. Low concentration means low blood supply. This electron microscopy studies documented that ATP molecules are low in muscles at rest and during contraction, thus confirming the impact of reduce capillary density on the amount of energy stoke. Reduced stroke shortens muscular performance and prolongs their recovery. These two processes are the real whole mark of fibromyalgia.

Fibromyalgia is a chronic vascular insufficiency syndrome created by unavoidable consequences of reduced capillary density leading to reduced fiber/capillary ratio that causes the fibromyalgia syndrome. All the peripheral and central nervous system (CNS) findings of the last 400 years in fibromyalgia can find their explanation in the capillary insufficiency pathogenesis. Capillaries insufficiency is a unified field explanation that reconciles the peripheral and CNS concepts of fibromyalgia.

The following fibromyalgia symptoms are the results of insufficient blood supply to fibromyalgia muscles, by insufficient capillaries numbers” chronic pain, symmetric distribution, morning stiffness, cold sensitivity, analgesic impact of heat, unexplained intense fatigue after moderate activity, longer recovery time from any activity, longer recovery time of any activity when compared to normal control, chronic fatigue of fibromyalgia, non-refreshing sleep, the tiredness on wakeup of fibromyalgia, and fibromyalgia fog. Even though all CNS symptoms can find their explanation as a secondary outcome of a chronic reduction in the fiber/capillary ratio of skeletal muscles, it opens the possibility that the fog itself is an independent CNS case of reduced fiber/capillary ratio of the brain. Fibromyalgia causes a part of the syndrome known as wide spread pain (WSP). It includes such syndromes as irritable bowel syndrome, temporomandibular joint (TMJ) syndrome, low back pain, and many others. Wide spread pain syndromes affects 15% of the world population. No logical or scientific explanations that cause these patients to suffer these symptoms have ever been found. Fibromyalgia represents 20% of those afflicted with WSP. Other WSP syndromes may be as well the consequence of a reduced capillaries density in their affected tissue.

This importance of reduced capillaries density in fibromyalgia has heretofore been ignored. Even though it is described and documented by many electron microscopy studies, its impact of insufficient blood supply by pathological fiber/capillaries ratio was never published. Such findings are also awaiting their studies in other WSP syndromes. Moreover, it gives a biological base to multiple epidemiological studies that documented the dramatic increase in fibromyalgia prevalence by aging. Eight recently internationally granted patents established that the complex aging process can be explained as the consequences of chronic progressive loss of body capillaries.

### Pharmaceutical Compositions for Treatment of Fibromyalgia and Associated Conditions

The pharmacological preparation can comprise a calcium channel blocker. The calcium channel blocker can be in a suitable nontoxic pharmacological carrier.

The pharmacological preparation can comprise an ACE inhibitor. The ACE inhibitor can be in a suitable nontoxic pharmacological carrier.

The pharmacological preparation can comprise an angiotensin receptor blocker. The angiotensin receptor blocker can be in a suitable nontoxic pharmacological carrier.

An effective amount for treatment of a symptom associated with fibromyalgia is administered. An amount of calcium channel blocker that is suitable for treatment by absorption through the epidermis of the affected areas is administered.

An effective amount for treatment of fibromyalgia is administered. An amount of ACE inhibitor that is suitable for treatment by absorption through the epidermis of the affected areas is administered.

An effective amount for treatment of fibromyalgia is administered. An amount of angiotensin receptor blocker that is suitable for treatment by absorption through the epidermis of the affected areas is administered.

Contact vasodilators (e.g., calcium channel blockers, ACE inhibitors, angiotensin receptor blockers, nitrates, alpha blockers, beta blockers, hydralazine, and/or angiotensin receptor-neprilysin inhibitors) are a new class of pharmaceutical medications that increase blood supply to the epidermis, e.g., in affected areas, which produces biological changes.

There is a need for a method to prevent, ameliorate or treat a symptom of fibromyalgia by increasing the blood supply to the epidermis and subcutaneous soft tissue, so as to produce biological changes that prevent, ameliorate, or treat a symptom of fibromyalgia.

A method is provided for preventing fibromyalgia by direct application to the epidermis of a vasodilator to prevent the formation and symptomatology of fibromyalgia.

Also, treatment is provided of fibromyalgia associated with conditions including, but not limited to, arthritis (e.g., rheumatoid arthritis), infection, post traumatic stress disorder, anxiety, depression, or physical inactivity.

In another embodiment, the vasodilators (e.g., calcium channel blockers, ACE inhibitors, angiotensin receptor blockers, nitrates, alpha blockers, beta blockers, hydralazine, and/or angiotensin receptor-neprilysin inhibitors) may be applied directly to the epidermis in an affected region to treat and heal the symptoms of fibromyalgia.

Calcium channel blockers are a new class of pharmaceutical drugs that disrupt the entry of calcium molecules through the L type voltage operated channels to cardiac muscle and blood vessels cells. The blockage of calcium entry causes the relief of arterial spasm.

Currently there are 70 pharmaceutical patented calcium channel blocker drugs that use this property to treat hypertension, angina pectoris and cardiac arrhythmia. The clinical indication for the therapeutic use of calcium channel blockers was therefore limited, until now, to the field of cardiovascular diseases only.

Calcium channel blockers were extensively studied but their ability to prevent and or to treat fibromyalgia remained heretofore unknown.

Accordingly, new uses are provided of topical calcium channel blockers for application to the epidermis and subcutaneous connective tissue in a region affected by fibromyalgia. The new use may be used for the prevention or treatment of fibromyalgia. No trial of topical calcium channel blockers for the prevention or treatment of fibromyalgia has heretofore been published.

Contact calcium channel blockers are a part of contact-vasodilators, a new class of medication. The use is provided of contact neo-vasodilators such as Nifedipine, a known calcium channel blocker used in the treatment of hypertension, for the prevention and treatment of fibromyalgia.

Nifedipine, Amlodipine, Felodipine, Isradipine, Nicardipine, Nisoldipine and Clevidipine are in a class of dihydropyridines calcium channel blockers. Verapamil and Diltiazem are non-dihydropyridines calcium channel blockers. When applied topically these are very effective drugs for the treatment or prevention of symptoms of fibromyalgia.

Inhibitors of angiotensin converting enzyme (ACE) can be employed as vasodilators. Angiotensin II is a chemical produced by the body that primarily circulates in the blood. It causes the muscles surrounding blood vessels to contract, thereby narrowing the vessels. Angiotensin II is formed from angiotensin I in the blood by the enzyme angiotensin converting enzyme (ACE). Angiotensin I in the blood is itself formed from angiotensinogen, a protein produced by the liver and released into the blood. Angiotensin converting enzyme inhibitors (ACE inhibitors) are medications that slow (inhibit) the activity of the enzyme ACE, which decreases the production of angiotensin II. As a result, blood vessels enlarge or dilate. ACE inhibitors include, but are not limited to benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik).

Angiotensin II receptor blockers (ARBs) help relax the blood vessels. Angiotensin II receptor blockers block the action of angiotensin II, allowing blood vessels to dilate. Angiotensin receptor blockers include, but are not limited to: azilsartan (Edarbi), candesartan (Atacand), eprosartan, irbesartan (Avapro), losartan (Cozaar), olmesartan (Benicar), telmisartan (Micardis), and valsartan (Diovan).

Other vasodilators are known in the art. These include, but are not limited to nitrates (nitroglycerin, isosorbide mononitrate and isosorbide dinitrate), Alpha blockers (doxazosin (Cardura), prazosin (Minipress), terazosin), Beta blockers (Acebutolol (Sectral), Atenolol (Tenormin), Bisoprolol fumarate (Zebeta), Carvedilol (Coreg)—Combined alpha/beta blocker, Esmilol (Brevibloc), Labetalol (Trandate, Normodyne)—Combined alpha/beta blocker, Metoprolol tartrate (Lopressor) and metoprolol succinate (Toprol-XL), Nadolol (Corgard), Nebivolol (Bystolic), Penbutolol sulfate (Levatol), Propranolol (Inderal), Sotalol (Betapace), HCTZ and bisoprolol (Ziac) is a beta blocker plus diuretic), Hydralazine, and angiotensin receptor-neprilysin inhibitors (ARNi) (Entresto, sacubitril/valsartan).

### Conditions Amenable to Treatment or Prevention

Compositions and methods are provided for the prevention or treatment of symptoms associated with fibromyalgia.

The spectrum of symptoms associated with fibromyalgia includes, but is not limited to, one or more of widespread pain, jaw pain and stiffness, pain and tiredness in the face muscles and adjacent fibrous tissues, stiff joints and muscles in the morning, headaches, irregular sleep patterns, irritable bowel syndrome, painful menstrual periods, tingling and numbness in the hands and feet, restless leg syndrome, sensitivity to cold or heat, difficulties with memory and concentration (“fibro-fog”), fatigue, problems with vision, nausea, pelvic and urinary problems, weight gain, dizziness, cold or flu-like symptoms, skin problems, chest symptoms, depression, anxiety, and breathing problems.

Application of vasodilators (e.g., calcium channel blockers, ACE inhibitors, angiotensin receptor blockers, nitrates, alpha blockers, beta blockers, hydralazine, and/or angiotensin receptor-neprilysin inhibitors), such as Nifedipine or other calcium channel blockers, which previously may have been used in the treatment of high blood pressure, in a pharmacological composition, in an effective amount, in a topical form, such as, but not limited to, a cream, ointment, gel base, foam, powder, spray, oil, liquid preparation or suspension, to the epidermis in affected areas can be employed to treat or prevent the symptoms of fibromyalgia.

Pharmacological compositions of the embodiments include but are not limited to one or more vasodilators (e.g., calcium channel blockers, ACE inhibitors, angiotensin receptor blockers, nitrates, alpha blockers, beta blockers, hydralazine, and/or angiotensin receptor-neprilysin inhibitors) and a suitable non toxic pharmaceutical carrier. The pharmaceutical composition in administered in an amount effective for treating fibromyalgia, e.g., an amount suitable for treatment by epidermal absorption in an affected area.

Fibromyalgia, associated symptoms, and treatment thereof are described in the following references, each of which is incorporated by reference herein in its entirety and each of which is hereby made a part of this specification: Clauw D J. Fibromyalgia: A clinical review. JAMA 2014; 311:1547; Goldenberg DL. Fibromyalgia syndrome. An emerging but controversial condition. JAMA 1987; 257:2782; Wolfe F, Clauw D J, Fitzcharles M A, et al. 2016 Revisions to the 2010/2011 fibromyalgia diagnostic criteria. Semin Arthritis Rheum 2016; 46:319; Wolfe F, Clauw D J, Fitzcharles M A, et al. 2016 Revisions to the 2010/2011 fibromyalgia diagnostic criteria. Semin Arthritis Rheum 2016; 46:319; Wolfe F, Ross K, Anderson J, et al. The prevalence and characteristics of fibromyalgia in the general population. Arthritis Rheum 1995; 38:19; Ting T V, Barnett K, Lynch-Jordan A, et al. 2010 American College of Rheumatology Adult Fibromyalgia Criteria for Use in an Adolescent Female Population with Juvenile Fibromyalgia. J Pediatr 2016; 169:181; Vincent A, Lahr B D, Wolfe F, et al. Prevalence of fibromyalgia: a population-based study in Olmsted County, Minn., utilizing the Rochester Epidemiology Project. Arthritis Care Res (Hoboken) 2013; 65:786; Walitt B, Nahin R L, Katz R S, et al. The Prevalence and Characteristics of Fibromyalgia in the 2012 National Health Interview Survey. PLoS One 2015; 10:e0138024; Wolfe F, Ross K, Anderson J, et al. The prevalence and characteristics of fibromyalgia in the general population. Arthritis Rheum; Jones GT, Atzeni F, Beasley M, et al. The prevalence of fibromyalgia in the general population: a comparison of the American College of Rheumatology 1990, 2010, and modified 2010 classification criteria. Arthritis Rheumatol 2015; 67:568; Walitt B, Nahin R L, Katz R S, et al. The Prevalence and Characteristics of Fibromyalgia in the 2012 National Health Interview Survey. PLoS One 2015; 10:e0138024.Walitt B, Nahin R L, Katz R S, et al. The Prevalence and Characteristics of Fibromyalgia in the 2012 National Health Interview; Arnold L M, Bennett R M, Crofford L J, et al. AAPT Diagnostic Criteria for Fibromyalgia. J Pain 2019; 20:611; Walitt B, Eko M, Khatiwada M, et al. Characterizing “fibrofog”: Subjective appraisal, objective performance, and task-related brain activity during a working memory task. Neuroimage Clin 2016; 11:173; Elkana O, Falcofsky A K, Shorer R, et al. Does the cognitive index of the symptom severity scale evaluate cognition? Data from subjective and objective cognitive measures in fibromyalgia. Clin Exp Rheumatol 2019; 37 Suppl 116:51; Wu Y L, Huang C J, Fang S C, et al. Cognitive Impairment in Fibromyalgia: A Meta-Analysis of Case-Control Studies. Psychosom Med 2018; 80:432; Fuller-Thomson E, Nimigon-Young J, Brennenstuhl S. Individuals with fibromyalgia and depression: findings from a nationally representative Canadian survey. Rheumatol Int 2012; 32:853; Loge-Hagen J S, Sele A, Juhl C, et al. Prevalence of depressive disorder among patients with fibromyalgia: Systematic review and meta-analysis. J Affect Disord 2019; 245:1098; de Tommaso M, Federici A, Serpino C, et al. Clinical features of headache patients with fibromyalgia comorbidity. J Headache Pain 2011; 12:629; Kücüken S, Genç E, Ylmaz H, et al. The prevalence of fibromyalgia and its relation with headache characteristics in episodic migraine. Clin Rheumatol 2013; 32:983; Wolfe F, Clauw D J, Fitzcharles M A, et al. The American College of Rheumatology preliminary diagnostic criteria for fibromyalgia and measurement of symptom severity. Arthritis Care Res (Hoboken) 2010; 62:600; Wolfe F, Clauw D J, Fitzcharles M A, et al. Fibromyalgia criteria and severity scales for clinical and epidemiological studies: a modification of the ACR Preliminary Diagnostic Criteria for Fibromyalgia. J Rheumatol 2011; 38:1113; Wolfe F, Clauw D J, Fitzcharles M A, et al. 2016 Revisions to the 2010/2011 fibromyalgia diagnostic criteria. Semin Arthritis Rheum 2016; 46:31; Sarzi-Puttini P, Atzeni F, Mease P J. Chronic widespread pain: from peripheral to central evolution. Best Pract Res Clin Rheumatol 2011; 25:133; Buskila D, Sarzi-Puttini P. Biology and therapy of fibromyalgia. Genetic aspects of fibromyalgia syndrome. Arthritis Res Ther 2006; Arnold L M, Hudson J I, Hess E V, et al. Family study of fibromyalgia. Arthritis Rheum 2004; 50:944; Limer K L, Nicholl B I, Thomson W, McBeth J. Exploring the genetic susceptibility of chronic widespread pain: the tender points in genetic association studies. Rheumatology (Oxford) 2008; 47:572; Staud R, Weyl E E, Riley J L 3rd, Fillingim R B. Slow temporal summation of pain for assessment of central pain sensitivity and clinical pain of fibromyalgia patients. PLoS One 2014; 9:e89086; Dadabhoy D, Crofford L J, Spaeth M, et al. Biology and therapy of fibromyalgia. Evidence-based biomarkers for fibromyalgia syndrome. Arthritis Res Ther 2008; 10:211; Jensen K B, Kosek E, Petzke F, et al. Evidence of dysfunctional pain inhibition in Fibromyalgia reflected in rACC during provoked pain. Pain 2009; 144:95; Harris R E, Clauw D J, Scott D J, et al. Decreased central mu-opioid receptor availability in fibromyalgia. J Neurosci 2007; 27:10000; Russell I J, Orr M D, Littman B, et al. Elevated cerebrospinal fluid levels of substance P in patients with the fibromyalgia syndrome. Arthritis Rheum 1994; 37:1593; Haas L, Portela L V, Bohmer A E, et al. Increased plasma levels of brain derived neurotrophic factor (BDNF) in patients with fibromyalgia. Neurochem Res 2010; 35:830; Giesecke T, Gracely R H, Williams D A, et al. The relationship between depression, clinical pain, and experimental pain in a chronic pain cohort. Arthritis Rheum 2005; 52:1577; Emad Y, Ragab Y, Zeinhom F, et al. Hippocampus dysfunction may explain symptoms of fibromyalgia syndrome. A study with single-voxel magnetic resonance spectroscopy. J Rheumatol 2008; 35:1371; Lutz J, Jager L, de Quervain D, et al. White and gray matter abnormalities in the brain of patients with fibromyalgia: a diffusion-tensor and volumetric imaging study. Arthritis Rheum 2008; 58:3960; Wood P B, Patterson J C 2nd, Sunderland J J, et al. Reduced presynaptic dopamine activity in fibromyalgia syndrome demonstrated with positron emission tomography: a pilot study. J Pain 2007; 8:51; Wood P B, Schweinhardt P, Jaeger E, et al. Fibromyalgia patients show an abnormal dopamine response to pain. Eur J Neurosci 2007; 25:3576; Sarzi-Puttini P, Atzeni F, Mease P J. Chronic widespread pain: from peripheral to central evolution. Best Pract Res Clin Rheumatol 2011; 25:133; Schmidt-Wilcke T, Clauw D J. Fibromyalgia: from pathophysiology to therapy. Nat Rev Rheumatol 2011; 7:518; Giesecke T, Gracely R H, Williams D A, et al. The relationship between depression, clinical pain, and experimental pain in a chronic pain cohort. Arthritis Rheum 2005; 52:1577; Kuchinad A, Schweinhardt P, Seminowicz D A, et al. Accelerated brain gray matter loss in fibromyalgia patients: premature aging of the brain? J Neurosci 2007; 27:4004; Burgmer M, Gaubitz M, Konrad C, et al. Decreased gray matter volumes in the cingulo-frontal cortex and the amygdala in patients with fibromyalgia. Psychosom Med 2009; 71:566; Hsu M C, Harris R E, Sundgren P C, et al. No consistent difference in gray matter volume between individuals with fibromyalgia and age-matched healthy subjects when controlling for affective disorder. Pain 2009; 143:262; Harris R E, Sundgren P C, Craig A D, et al. Elevated insular glutamate in fibromyalgia is associated with experimental pain. Arthritis Rheum 2009; 60:3146; Cagnie B, Coppieters I, Denecker S, et al. Central sensitization in fibromyalgia? A systematic review on structural and functional brain MRI. Semin Arthritis Rheum 2014; 44:68; Moldofsky H. The significance of dysfunctions of the sleeping/waking brain to the pathogenesis and treatment of fibromyalgia syndrome. Rheum Dis Clin North Am 2009; 35:275; Roizenblatt S, Neto N S, Tufik S. Sleep disorders and fibromyalgia. Curr Pain Headache Rep 2011; 15:347; Rizzi M, Sarzi-Puttini P, Atzeni F, et al. Cyclic alternating pattern: a new marker of sleep alteration in patients with fibromyalgia? J Rheumatol 2004; 31:1193; McBeth J, Lacey R J, Wilkie R. Predictors of new-onset widespread pain in older adults: results from a population-based prospective cohort study in the U K. Arthritis Rheumatol 2014; 66:757; Yeung W K, Morgan K, Mckenna F. Comparison of sleep structure and psychometric profiles in patients with fibromyalgia, osteoarthritis and healthy controls. J Sleep Res 2018; 27:290; Weissbecker I, Floyd A, Dedert E, et al. Childhood trauma and diurnal cortisol disruption in fibromyalgia syndrome. Psychoneuroendocrinology 2006; 31:312; McLean S A, Williams D A, Harris R E, et al. Momentary relationship between cortisol secretion and symptoms in patients with fibromyalgia. Arthritis Rheum 2005; 52:3660; Wingenfeld K, Heim C, Schmidt I, et al. HPA axis reactivity and lymphocyte glucocorticoid sensitivity in fibromyalgia syndrome and chronic pelvic pain. Psychosom Med 2008; 70:65; El Maghraoui A, Tellal S, Achemlal L, et al. Bone turnover and hormonal perturbations in patients with fibromyalgia. Clin Exp Rheumatol 2006; 24:428; Solano C, Martinez A, Becerril L, et al. Autonomic dysfunction in fibromyalgia assessed by the Composite Autonomic Symptoms Scale (COMPASS). J Clin Rheumatol 2009; 15:172; Maekawa K, Twe C, Lotaif A, et al. Function of beta-adrenergic receptors on mononuclear cells in female patients with fibromyalgia. J Rheumatol 2003; 30:364; Kadetoff D, Kosek E. The effects of static muscular contraction on blood pressure, heart rate, pain ratings and pressure pain thresholds in healthy individuals and patients with fibromyalgia. Eur J Pain 2007; 11:39; Riva R, Mork P J, Westgaard R H, et al. Catecholamines and heart rate in female fibromyalgia patients. J Psychosom Res 2012; 72:51; Kadetoff D, Kosek E. Evidence of reduced sympatho-adrenal and hypothalamic-pituitary activity during static muscular work in patients with fibromyalgia. J Rehabil Med 2010; 42:765; Lerma C, Martinez A, Ruiz N, et al. Nocturnal heart rate variability parameters as potential fibromyalgia biomarker: correlation with symptoms severity. Arthritis Res Ther 2011; 13:R185; Maia M M, Gualano B, Sá-Pinto A L, et al. Juvenile fibromyalgia syndrome: Blunted heart rate response and cardiac autonomic dysfunction at diagnosis. Semin Arthritis Rheum 2016; 46:338; Nishikai M, Tomomatsu S, Hankins R W, et al. Autoantibodies to a 68/48 kDa protein in chronic fatigue syndrome and primary fibromyalgia: a possible marker for hypersomnia and cognitive disorders. Rheumatology (Oxford) 2001; 40:806; Bazzichi L, Rossi A, Massimetti G, et al. Cytokine patterns in fibromyalgia and their correlation with clinical manifestations. Clin Exp Rheumatol 2007; 25:225; Uçeyler N, Häuser W, Sommer C. Systematic review with meta-analysis: cytokines in fibromyalgia syndrome. BMC Musculoskelet Disord 2011; 12:245; Uçeyler N, Hauser W, Sommer C. Systematic review with meta-analysis: cytokines in fibromyalgia syndrome. BMC Musculoskelet Disord 2011; 12:245; Staud R. Peripheral pain mechanisms in chronic widespread pain. Best Pract Res Clin Rheumatol 2011; 25:155; Uçeyler N, Zeller D, Kahn A K, et al. Small fibre pathology in patients with fibromyalgia syndrome. Brain 2013; 136:1857; Oaklander A L, Herzog Z D, Downs H M, Klein M M. Objective evidence that small-fiber polyneuropathy underlies some illnesses currently labeled as fibromyalgia. Pain 2013; 154:2310; Giannoccaro M P, Donadio V, Incensi A, et al. Small nerve fiber involvement in patients referred for fibromyalgia. Muscle Nerve 2014; 49:757; Caro X J, Winter E F. Evidence of abnormal epidermal nerve fiber density in fibromyalgia: clinical and immunologic implications; Gerdle B, Forsgren M F, Bengtsson A, et al. Decreased muscle concentrations of ATP and PCR in the quadriceps muscle of fibromyalgia patients—a 31P-MRS study. Eur J Pain 2013; 17:1205; Srikuea R, Symons T B, Long D E, et al. Association of fibromyalgia with altered skeletal muscle characteristics which may contribute to postexertional fatigue in postmenopausal women. Arthritis Rheum 2013; 65:519; Hughes G, Martinez C, Myon E, et al. The impact of a diagnosis of fibromyalgia on health care resource use by primary care patients in the U K: an observational study based on clinical practice. Arthritis Rheum 2006; 54:177; Annemans L, Wessely S, Spaepen E, et al. Health economic consequences related to the diagnosis of fibromyalgia syndrome; Pfeiffer A, Thompson J M, Nelson A, et al. Effects of a 1.5-day multidisciplinary outpatient treatment program for fibromyalgia: a pilot study. Am J Phys Med Rehabil 2003; 82:186; Busch A J, Webber S C, Richards R S, et al. Resistance exercise training for fibromyalgia. Cochrane Database Syst Rev 2013; Bidonde J, Busch A J, Webber S C, et al. Aquatic exercise training for fibromyalgia. Cochrane Database Syst Rev 2014; Hauser W, Klose P, Langhorst J, et al. Efficacy of different types of aerobic exercise in fibromyalgia syndrome: a systematic review and meta-analysis of randomised controlled trials. Arthritis Res Ther 2010; 12:R79; Hauser W, Wolfe F, Tolle T, et al. The role of antidepressants in the management of fibromyalgia syndrome: a systematic review and meta-analysis. CNS Drugs 2012; 26:297; Wolfe F, Anderson J, Harkness D, et al. Health status and disease severity in fibromyalgia: results of a six-center longitudinal study; Walitt B, Fitzcharles M A, Hassett A L, et al. The longitudinal outcome of fibromyalgia: a study of 1555 patients. J Rheumatol 2011; 38:2238; and White K P, Speechley M, Harth M, Ostbye T. Comparing self-reported function and work disability in 100 community cases of fibromyalgia syndrome versus controls in London, Ontario: the London Fibromyalgia Epidemiology Study. Arthritis Rheum 1999; 42:76.

Compositions including one or more vasodilators (e.g., calcium channel blockers, ACE inhibitors, angiotensin receptor blockers, nitrates, alpha blockers, beta blockers, hydralazine, and/or angiotensin receptor-neprilysin inhibitors), optionally in combination with conventional therapies, and associated methods for treatment of fibromyalgia and related symptoms are provided.

Some embodiments relate to a pharmaceutical composition and method of treatment using the pharmaceutical composition, wherein the pharmaceutical composition comprises at least one calcium channel blocker, for example, a calcium channel blocker selected from the group consisting of amlodipine (Norvasc), diltiazem (Cardizem LA, Tiazac), felodipine (Plendil), isradipine (Dynacirc), nifedipine (Adalat, Procardia), nicardipine (Cardene), nimodipine (Nimotop), nisoldipine (Sular), verapamil (Covera-HS, Verelan PM, Calan), verapamil, diltiazem and nicardipine (Cardene IV). Some embodiments relate to a pharmaceutical composition and method of treatment using the pharmaceutical composition, wherein the pharmaceutical composition comprises at least one ACE inhibitors, for example at least one ACE inhibitor selected from the group consisting of benazepril (Lotensin), captopril (Capoten), enalapril (Vasotec, Epaned, Lexxel), fosinopril (Monopril), lisinopril (Prinivil), moexipril (Univasc), perindopril (Aceon), quinapril (Accupril), ramipril (Altace), and trandolapril (Mavik). Some embodiments relate to a pharmaceutical composition and method of treatment using the pharmaceutical composition, wherein the pharmaceutical composition comprises at least one angiotensin receptor blocker, for example at least one angiotensin receptor blocker selected from the group consisting of azilsartan (Edarbi), candesartan (Atacand), eprosartan, irbesartan (Avapro), losartan (Cozaar), olmesartan (Benicar), telmisartan (Micardis), and valsartan (Diovan). In certain embodiments, the pharmaceutical composition is in a form suitable for topical administration, e.g., to epidermis, however other routes of administration are also considered that involve contact of the vasodilator to the tissue to be treated.

The pharmaceutical compositions for treatment of fibromyalgia can further comprise other pharmaceutically active ingredients, or can be administered in conjunction with a conventional treatment for fibromyalgia, e.g., nonsteroidal antiinflammatory drugs (NSAIDs, e.g., ibuprofen, aspirin, naproxen sodium, oxaprozin, etodolac, indomethacin, naproxen, nabumetone, diclofenac, or vimovo (naproxen/esomeprazole)) such as antidepressants (e.g., duloxetine, Cymbalta, milnacipran, or Savella), antiseizure drugs (e.g., gabapentin, Neurontin, pregabalin, or Lyrica).

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, and phosphates.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, tocopherols, carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more pharmacologically active ingredients (e.g. calcium channel blockers) disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles, and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

It is to be understood that where compounds disclosed herein (e.g., calcium channel blockers, ACE inhibitors, angiotensin receptor blockers, nitrates, alpha blockers, beta blockers, hydralazine, and/or angiotensin receptor-neprilysin inhibitors) have unfilled valencies, then the valencies are to be filled with hydrogens or isotopes thereof, e.g., hydrogen-1 (protium) and hydrogen-2 (deuterium).

It is understood that the compounds described herein (e.g., calcium channel blockers, ACE inhibitors, angiotensin receptor blockers, nitrates, alpha blockers, beta blockers, hydralazine, and/or angiotensin receptor-neprilysin inhibitors) can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein may include crystalline forms (also known as polymorphs, which include the different crystal packing arrangements of the same elemental composition of a compound), amorphous phases, salts, solvates, and hydrates, e.g., vasodilators. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein (e.g., calcium channel blockers, ACE inhibitors, angiotensin receptor blockers, nitrates, alpha blockers, beta blockers, hydralazine, and/or angiotensin receptor-neprilysin inhibitors) may exist in unsolvated as well as solvated forms. In general, the solvated forms are considered equivalent to the unsolvated forms for the purposes of the compounds and methods provided herein.

Where a range of values is provided, it is understood that the upper and lower limit, and any intervening value between the upper and lower limit of the range is included.

Any percentages, ratios or other quantities referred to herein are on a weight basis, unless otherwise indicated.

### Pharmaceutical Compositions

The vasodilators (e.g., calcium channel blockers, ACE inhibitors, angiotensin receptor blockers, nitrates, alpha blockers, beta blockers, hydralazine, and/or angiotensin receptor-neprilysin inhibitors) can be prepared by any suitable method known to those in the art. For representative methods, see, for example, Francis A. Carey et al., Advanced Organic Chemistry: Part B: Reaction and Synthesis (5^{th} Ed. 2005).

Formulations including a vasodilator (e.g., a calcium channel blocker, ACE inhibitor and/or angiotensin receptor blocker) and at least one excipient are provided. It is generally preferred to administer the compounds of the embodiments in topical formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the vasodilator (e.g., a calcium channel blocker, ACE inhibitor and/or angiotensin receptor blocker) used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The compositions described herein are suitable for use in treatment or prevention of fibromyalgia or associated symptoms. The compositions are suitable for use in any patient where treatment or prevention of fibromyalgia is desirable.

The vasodilator (e.g., a calcium channel blocker, ACE inhibitor and/or angiotensin receptor blocker) can be employed in various types of formulations. Topical formulations including one or more vasodilators in combination with at least one excipient are provided. Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical formulations for application to tissue or mucous membranes. The formulation can be provided as an aqueous formulation, or in an anhydrous formulation which may prevent water-based irritant contact dermatitis or stinging sensation upon application. In another embodiment, the composition is formulated such that preservatives need not be employed (e.g., a preservative-free formulation) so as to avoid skin irritation associated with certain preservatives.

To facilitate application, the composition may be provided as an ointment, an oil, a lotion, a paste, a powder, a gel, or a cream. The composition may also include additional ingredients such as a protective agent, an emollient, a humectant, an antibiotic agent, an antifungal agent, an antiviral agent, an antiprotozoal agent, an anesthetic agent, a steroidal anti-inflammatory agent, a non-steroidal anti-inflammatory agent, an antipruritic agent, an antioxidant agent, an anti-histamine agent, a vitamin or vitamin complex, a hormone, an anti-skin atrophy agent, and combinations thereof. In a further embodiment, the composition may avoid animal or cellular-based materials to avoid irritation. The composition can be applied to the dermis, or to mucous membranes.

Methods of using topical vasodilator formulations are provided. The compositions may also be applied to treat fibromyalgia and/or associated symptoms.

Some embodiments include administering vasodilator (e.g., a calcium channel blocker, ACE inhibitor and/or angiotensin receptor blocker) compositions provided herein in topical formulations; however, other routes of administration are also contemplated (e.g., mucosal, subdermal, oral, or the like). Contemplated routes of administration include but are not limited to topical, mucosal, and subcutaneous. Suitable liquid forms include suspensions, emulsions, solutions, and the like. Unit dosage forms can also be provided, e.g., individual packets with a premeasured amount of the formulation, configured for administration to the tissue on a predetermined schedule (e.g., daily, weekly, etc.). Unit dosage forms configured for administration twice a day can be employed; however, in certain embodiments it can be desirable to configure the unit dosage form for administration once a day, four times a day, or more, or once every other day, every three days, weekly, or less, or on an as-needed basis.

In some embodiments, the topical and other formulations typically comprise from about 0.001 wt. % or less to about 50 wt. % or more of active ingredient, such as the vasodilator (e.g., a calcium channel blocker, ACE inhibitor and/or angiotensin receptor blocker), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Compositions and formulations for topical administration can include transdermal patches, ointments, lotions, creams, gels, drops, sprays, liquids, aerosols, and powders. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the tissue surface, liquid in the container is transferred in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

A topical formulation can be provided in a form of a carrier containing the vasodilator, e.g., 50 ppm or less to 1000, 5000, 10000, 50000, 100000, 500000 ppm or more of the vasodilator. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % or 20 wt. % of the vasodilator. The amount of vasodilator in the base can be adjusted up or down.

Liquids and gels containing the vasodilator, optionally with other components as described herein, can be prepared using techniques as are known in the art for preparing topical compositions. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to skin or mucous membranes.

Penetration enhancers can be employed to enhance penetration of the vasodilator into tissue, and to provide a silky feel to formulations. Typical amounts when employed in topical formulations are from 1% by weight to 4% by weight. Typical amounts for anti-irritation agents when employed in topical formulations are from 1% by weight to 4% by weight. Typical amounts for anti-inflammatory agents when employed in topical formulations are from 1% by weight to 4% by weight. Typical amounts for anti-inflammatory agents when employed in topical formulations are from 0.1% by weight to 2% by weight.

In some embodiments, the vasodilator can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the vasodilator as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the vasodilator into the skin or mucous membranes.

The pharmaceutical excipients used in the topical preparations of the vasodilator compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the vasodilator compositions as described herein may be dissolved or dispersed in a hydrophobic oil phase, and the oil phase may then be emulsified in an aqueous phase comprising water, alone or in combination with lower alcohols, glycerin, and/or glycols. In certain embodiments water is present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carragenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the vasodilator compositions as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the vasodilator compositions as described herein.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The vasodilator compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the vasodilator formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The vasodilator formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

### Kits for Administration of Compositions

Some embodiments of the methods and compositions provided herein include kits comprising vasodilators provided herein. In some embodiments, kits can be provided to an administering physician, other health care professional, a patient, or a caregiver. In some embodiments, a kit comprises a container which contains the vasodilator in a suitable topical formulation, and instructions for administering the composition to a subject. The kit can optionally also contain one or more additional therapeutic or other agents. The kit may contain the vasodilator in bulk form, or can contain separate doses of the vasodilator for serial or sequential administration. The kit can optionally contain one or more diagnostic tools, administration tools, and/or instructions for use. The kit can contain suitable delivery devices, such as, syringes, pump dispensers, wands, single dose packets, and the like, along with instructions for administering the vasodilator compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

The topical formulation, in addition to the vasodilator, can contain other ingredients.

While topical administration of the vasodilator disclosed herein can advantageously be employed, in certain embodiments other routes of administration are also contemplated.

The vasodilator compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The vasodilator compositions disclosed herein may be manufactured into administrable forms by a process that is itself known, e.g., by means of conventional mixing, dissolving, granulating, dragee-making, levigating, emulsifying, encapsulating, entrapping, tableting, or extracting processes.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, the vasodilator may be combined as the active ingredient in intimate admixture with a pharmaceutical carrier according to conventional pharmaceutical compounding techniques. The excipients are preferably minimized so as to ensure administration of an appropriate amount of vasodilator in a compact format. The carrier can take a wide variety of forms depending on the form of preparation desired for administration. Thus, the vasodilator compositions provided herein can be presented as discrete units suitable for administration each containing a predetermined amount of the active ingredient. Further, the vasodilator compositions can be presented as an oil, a powder, as granules, as a solution, as a suspension in an aqueous liquid, as a non-aqueous liquid, as an oil-in-water emulsion, or as a water-in-oil liquid emulsion, similar to the topical formulations described elsewhere herein, but using components suitable for human consumption. In addition to the common dosage forms set out above, the vasodilator compositions provided herein can also be administered by controlled release and/or delivery devices. The vasodilator compositions can be prepared by any of the methods of pharmacy. In general, such methods include a step of bringing into association the active ingredient with the carrier that constitutes one or more necessary ingredients. In general, the vasodilator compositions are prepared by uniformly and intimately admixing the vasodilator ingredient(s) with liquid carriers or finely divided solid carriers or both. The product can then be conveniently shaped into the desired presentation.

A vasodilator formulation may also be administered in a local manner, for example, via injection of the vasodilator composition directly into a target area, e.g., in a depot or sustained release formulation in skin tissue. Furthermore, a targeted drug delivery system for the vasodilator may be used, for example, in a liposome coated with a tissue specific antibody.

The vasodilator compositions may contain the vasodilator in an amount effective for the desired therapeutic effect. In some embodiments, the vasodilator compositions are in a unit dosage form and comprise from about 0.1 mg or less to about 5000 mg or more of vasodilator per unit dosage form. In further embodiments, the vasodilator compositions comprise from about 1 to about 500 mg per unit dosage form or from about 500 to 5000 mg per unit dosage form of vasodilator. Such amounts can be selected depending upon the vasodilator employed. Such dosage forms may be solid, semisolid, liquid, an emulsion, or adapted for delivery via aerosol or the like.

The carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Vasodilator compositions provided herein can be prepared as solutions or suspensions of the vasodilator in water or nonaqueous liquids. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Vasodilator compositions provided herein suitable for injectable use include sterile aqueous solutions or dispersions. Furthermore, the vasodilator compositions can be in the form of sterile powders for the extemporaneous preparation of such sterile injectable solutions or dispersions. The vasodilator compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Contemplated herein are vasodilator compositions including one or more vasodilators as described herein in combination with at least one additional active agent, e.g., an antibiotic. The vasodilator and the at least one additional active agent(s) may be present in a single formulation or in multiple formulations provided together, or may be unformulated. In some embodiments, the vasodilator can be administered with one or more additional agents together in a single composition. For example, the vasodilator can be administered in one composition, and at least one of the additional agents can be administered in a second composition. In a further embodiment, the vasodilator and the at least one additional active agent(s) are co-packaged in a kit. For example, a drug manufacturer, a drug reseller, a physician, a compounding shop, or a pharmacist can provide a kit comprising the vasodilator in combination with another product or component for delivery to a patient. Such additional components can include NSAIDs, antidepressants, and/or antiseizure drugs as described elsewhere herein, or the like.

Some embodiments described herein relate to compositions of vasodilator, which can include a therapeutically effective amount of the vasodilator described herein and a pharmaceutically acceptable carrier, diluent, excipient or combination thereof. The vasodilator composition can include the vasodilator in an amount for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

Eight recently internationally granted patents established that the complex aging process can be explained as the consequences of chronic progressive loss of body capillaries. The contents of these publications are hereby incorporated by reference in their entireties Ücyler N, Zeller D, Kahn A K, et al. Small fiber pathology in patients with fibromyalgia syndrome. Brain 2013; 136: 1857; Oaklander A L, Herzog Z D, Downs H M, Kelin M M. Objective evidence that small fiber polyneuropathy underlies some illnesses currently labelled as fibromyalgia. Pain 2013; 154:2310; Giannocarro M P, Donadio V, Incensi A, et al. Small nerve fiber involvement in patients referred for fibromyalgia. Muscle Nerve 2014; 49:757; Caro X J, Winter E F. Evidence of abnormal epidermal nerve fiber density in fibromyalgia; clinical and immunologic implications. Arthritis Rheumatology 2014; 66:1945; Grayston R, Czanner G, Elhadd K, et al. A systematic review and meta-analysis of the prevalence of small fiber pathology in fibromyalgia; Implications for a new paradigm in Fibromyalgia etiopathogenesis. Semin Arthritis Rheum 2019; 48:933; Lawson V H, Grewal J, Hackshaw K V, et al. Fibromyalgia syndrome and small fiber, early or mild sensory polyneuropathy. Muscle Nerve 2018; 58:625; Harte S E, Clauw D J, Hayes J M, et al. Imaging signatures of altered brain responses in small-fiber neuropathy; reduced functional connectivity of the limbic system after peripheral nerve degeneration. Pain 2015; 156:904; Gerdle B, Forsgren M F, Bengtsson A, et al. Decreased muscle concentrations of ATP and PCR in quadriceps muscles of fibromyalgia patients—a 31P-MRS study. Eur J Pain 2013; 17:1205; and Srikuea R, Symons T B, Long D E, et al. Association of Fibromyalgia with altered skeletal muscle characteristics which may contribute to postexertional fatigue in postmenopausal women. Arthritis Rheum 2013; 65:519.

Publications related to compositions as disclosed herein for promoting neocapillary formation include the following, each of which is incorporated by reference herein in its entirety: WO 2019/226548; WO 2019/226542; WO 2020/123312; WO 2020/123852; WO 2020/139784; WO 2020/142290; WO 2020/154304; and WO 2020/118254.

### EXAMPLES

The following prophetic examples are of cases that address the painful experiences associated with fibromyalgia. The examples also describe a new, safe, simple and until now, an unknown pharmaceutical solution to the problem of fibromyalgia syndrome: the novel use of vasodilators such as calcium channel blockers, ACE inhibitors, and angiotensin receptor blockers. Vasodilators are not analgesics, but they resolve or mitigate the pain associated with fibromyalgia. Vasodilators are among the safest, widely used medication in the world.

### Example 1

A patient is diagnosed with fibromyalgia, and suffers from pain in soft tissue regions and myofacial regions. Daily use of nifedipine in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 2

A patient is diagnosed with fibromyalgia, and suffers from pain in soft tissue regions and myofacial regions. Daily use of Amlodipine in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 3

A patient is diagnosed with fibromyalgia, and suffers from pain in soft tissue regions and myofacial regions. Daily use of enalapril in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 4

A patient is diagnosed with fibromyalgia, and suffers from pain in soft tissue regions and myofacial regions. Daily use of lisinopril in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 5

A patient is diagnosed with fibromyalgia, and suffers from pain in soft tissue regions and myofacial regions. Daily use of losartan in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 6

A patient is diagnosed with fibromyalgia, and suffers from pain in soft tissue regions and myofacial regions. Daily use of irbesartan in a topical form is prescribed for application to skin adjacent to regions where pain is experienced. The patient reports reduction in pain levels in the treated regions. After several months, treatment is halted. Weeks later, the patient reports that the reduction in pain levels has been maintained.

### Example 7

A patient is diagnosed with a traumatic injury, arthritis (e.g., rheumatoid arthritis), or an autoimmune disorder (e.g., lupus), but does not exhibit symptoms of fibromyalgia. Daily use of nifedipine in a topical form is prescribed for application to skin in areas where fibromyalgia is commonly experiences (e.g., in regions of soft tissue or myofacial regions). The patient remains free of symptoms of fibromyalgia in the treated regions. After several months, treatment is halted. The patient remains free of symptoms of fibromyalgia in the previously treated regions weeks later.

### Example 8

This case involves the use of a different calcium channel blocker. A patient is diagnosed with a traumatic injury, arthritis (e.g., rheumatoid arthritis), or an autoimmune disorder (e.g., lupus), but does not exhibit symptoms of fibromyalgia. Daily use of Amlodipine in a topical form is prescribed for application to skin in areas where fibromyalgia is commonly experiences (e.g., in regions of soft tissue or myofacial regions). The patient remains free of symptoms of fibromyalgia in the treated regions for the duration of the treatment. After several months, treatment is halted. The patient remains free of symptoms of fibromyalgia in the previously treated regions weeks later.

### Example 9

This case involves the use of a different calcium channel blocker. A patient is diagnosed with a traumatic injury, arthritis (e.g., rheumatoid arthritis), or an autoimmune disorder (e.g., lupus), but does not exhibit symptoms of fibromyalgia. Daily use of enalapril in a topical form is prescribed for application to skin in areas where fibromyalgia is commonly experiences (e.g., in regions of soft tissue or myofacial regions). The patient remains free of symptoms of fibromyalgia in the treated regions for the duration of the treatment. After several months, treatment is halted. The patient remains free of symptoms of fibromyalgia in the previously treated regions weeks later.

### Example 10

This case involves the use of a different calcium channel blocker. A patient is diagnosed with a traumatic injury, arthritis (e.g., rheumatoid arthritis), or an autoimmune disorder (e.g., lupus), but does not exhibit symptoms of fibromyalgia. Daily use of lisinopril in a topical form is prescribed for application to skin in areas where fibromyalgia is commonly experiences (e.g., in regions of soft tissue or myofacial regions). The patient remains free of symptoms of fibromyalgia in the treated regions for the duration of the treatment. After several months, treatment is halted. The patient remains free of symptoms of fibromyalgia in the previously treated regions weeks later.

### Example 11

This case involves the use of a different calcium channel blocker. A patient is diagnosed with a traumatic injury, arthritis (e.g., rheumatoid arthritis), or an autoimmune disorder (e.g., lupus), but does not exhibit symptoms of fibromyalgia. Daily use of losartan in a topical form is prescribed for application to skin in areas where fibromyalgia is commonly experiences (e.g., in regions of soft tissue or myofacial regions). The patient remains free of symptoms of fibromyalgia in the treated regions for the duration of the treatment. After several months, treatment is halted. The patient remains free of symptoms of fibromyalgia in the previously treated regions weeks later.

### Example 12

This case involves the use of a different calcium channel blocker. A patient is diagnosed with a traumatic injury, arthritis (e.g., rheumatoid arthritis), or an autoimmune disorder (e.g., lupus), but does not exhibit symptoms of fibromyalgia. Daily use of irbesartan in a topical form is prescribed for application to skin in areas where fibromyalgia is commonly experiences (e.g., in regions of soft tissue or myofacial regions). The patient remains free of symptoms of fibromyalgia in the treated regions for the duration of the treatment. After several months, treatment is halted. The patient remains free of symptoms of fibromyalgia in the previously treated regions weeks later.

### Example 13

A patient is diagnosed with fibromyalgia. Daily use of nifedipine in a topical form is prescribed for application to skin adjacent to regions where pain is experienced. Neo capillary formation is observed after treatment.

### Example 14

A patient is diagnosed with fibromyalgia. Daily use of Amlodipine in a topical form is prescribed for application to skin adjacent to regions where pain is experienced. Neo capillary formation is observed after treatment.

### Example 15

A patient is diagnosed with fibromyalgia. Daily use of enalapril in a topical form is prescribed for application to skin adjacent to regions where pain is experienced. Neo capillary formation is observed after treatment.

### Exemplary Compositions, Methods and Uses

Pharmaceutical Composition 1: A pharmaceutical composition for the treatment or prophylaxis of fibromyalgia syndrome, comprising: at least one vasodilator; and at least one pharmaceutical excipient.

Pharmaceutical Composition 2: Pharmaceutical Composition 1, in a form adapted for topical administration to an epidermis.

Pharmaceutical Composition 3: Pharmaceutical Composition 1 or 2, wherein the form is selected from the group consisting of a cream, a lotion, an ointment, a gel base, a foam, a powder, an aerosol spray, an oil, a liquid, and a suspension.

Pharmaceutical Composition 4: Any one of Pharmaceutical Compositions 1 through 3, wherein the vasodilator is a calcium channel blocker.

Pharmaceutical Composition 5: Pharmaceutical Composition 4, wherein the at least one calcium channel blocker is a dihydropyridine selected from the group consisting of nifedipine, isradipine, felodipine, amlodipine, nicardipine, and clevidipine.

Pharmaceutical Composition 6: Pharmaceutical Composition 4, wherein the at least one calcium channel blocker is a non dihydropyridine selected from the group consisting of verapamil and diltiazem.

Pharmaceutical Composition 7: Any one of Pharmaceutical Compositions 1 through 3, wherein the vasodilator is an ACE inhibitor.

Pharmaceutical Composition 8: Pharmaceutical Composition 7, wherein the ACE inhibitor is selected from the group consisting of benazepril, captopril, enalapril, fosinopril, lisinopril, moexipril, perindopril, quinapril, ramipril, and trandolapril.

Pharmaceutical Composition 9: Any one of Pharmaceutical Compositions 1 through 3, wherein the vasodilator is an angiotensin receptor blocker.

Pharmaceutical Composition 10: Pharmaceutical Composition 9, wherein the angiotensin receptor blocker is selected from the group consisting of azilsartan, candesartan, eprosartan, irbesartan, losartan, olmesartan, telmisartan, and valsartan.

Pharmaceutical Composition 11: Any one of Pharmaceutical Compositions 1 through 3, wherein the vasodilator is a nitrate.

Pharmaceutical Composition 12: Pharmaceutical Composition 11, wherein the nitrate is selected from the group consisting of nitroglycerin, isosorbide mononitrate and isosorbide dinitrate.

Pharmaceutical Composition 13: Any one of Pharmaceutical Compositions 1 through 3, wherein the vasodilator is an alpha blocker.

Pharmaceutical Composition 14: Pharmaceutical Composition 13, wherein the alpha blocker is selected from the group consisting of doxazosin, prazosin, and terazosin.

Pharmaceutical Composition 15: Any one of Pharmaceutical Compositions 1 through 3, wherein the vasodilator is a beta blocker.

Pharmaceutical Composition 16: Pharmaceutical Composition 15, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 17: Any one of Pharmaceutical Compositions 1 through 3, wherein the vasodilator is hydralazine.

Pharmaceutical Composition 18: Any one of Pharmaceutical Compositions 1 through 3, wherein the vasodilator is an angiotensin receptor-neprilysin inhibitor.

Pharmaceutical Composition 19: Pharmaceutical Composition 18, wherein the angiotensin receptor-neprilysin inhibitor is sacubitril/valsartan.

Method 20: A method for the treatment or prophylaxis of fibromyalgia in a patient in need thereof, comprising: administering an effective amount of the pharmaceutical composition according to any one of Pharmaceutical Compositions 1 through 19 to a patient in need thereof, whereby a symptom of fibromyalgia, is treated or prevented.

Method 21: Method 20, the symptom is pain.

Method 22: Method 21, wherein the symptom is myofacial pain.

Method 23: Method 21, wherein the symptom is soft tissue pain.

Pharmaceutical Composition 24: A pharmaceutical composition for the promotion of neo capillary formation, comprising: at least one vasodilator; and at least one pharmaceutical excipient.

Pharmaceutical Composition 25: Pharmaceutical Composition 24, in a form adapted for topical administration to an epidermis.

Pharmaceutical Composition 26: Pharmaceutical Composition 24 or 25, wherein the form is selected from the group consisting of a cream, a lotion, an ointment, a gel base, a foam, a powder, an aerosol spray, an oil, a liquid, and a suspension.

Pharmaceutical Composition 27: Any one of Pharmaceutical Compositions 24 through 26, wherein the vasodilator is a calcium channel blocker.

Pharmaceutical Composition 28: Pharmaceutical Composition 27, wherein the at least one calcium channel blocker is a dihydropyridine selected from the group consisting of nifedipine, isradipine, felodipine, amlodipine, nicardipine, and clevidipine.

Pharmaceutical Composition 29: Pharmaceutical Composition 27 wherein the at least one calcium channel blocker is a non dihydropyridine selected from the group consisting of verapamil and diltiazem.

Pharmaceutical Composition 30: Any one of Pharmaceutical Compositions 24 through 26, wherein the vasodilator is an ACE inhibitor.

Pharmaceutical Composition 31: Pharmaceutical Composition 30, wherein the ACE inhibitor is selected from the group consisting of benazepril, captopril, enalapril, fosinopril, lisinopril, moexipril, perindopril, quinapril, ramipril, and trandolapril.

Pharmaceutical Composition 32: Any one of Pharmaceutical Compositions 24 through 26, wherein the vasodilator is an angiotensin receptor blocker.

Pharmaceutical Composition 33: Pharmaceutical Composition 32, wherein the angiotensin receptor blocker is selected from the group consisting of azilsartan, candesartan, eprosartan, irbesartan, losartan, olmesartan, telmisartan, and valsartan.

Pharmaceutical Composition 34: Any one of Pharmaceutical Compositions 24 through 26, wherein the vasodilator is a nitrate.

Pharmaceutical Composition 35: Pharmaceutical Composition 34, wherein the nitrate is selected from the group consisting of nitroglycerin, isosorbide mononitrate and isosorbide dinitrate.

Pharmaceutical Composition 36: Any one of Pharmaceutical Compositions 24 through 26, wherein the vasodilator is an alpha blocker.

Pharmaceutical Composition 37: Pharmaceutical Composition 36, wherein the alpha blocker is selected from the group consisting of doxazosin, prazosin, and terazosin.

Pharmaceutical Composition 38: Any one of Pharmaceutical Compositions 24 through 26, wherein the vasodilator is a beta blocker.

Pharmaceutical Composition 39: Pharmaceutical Composition 38, wherein the beta blocker is selected from the group consisting of acebutolol, atenolol, bisoprolol fumarate, carvedilol, esmilol, labetalol, metoprolol tartrate, metoprolol succinate, nadolol, nebivolol, penbutolol sulfate, propranolol, sotalol, hydrochlorothiazide, and bisoprolol.

Pharmaceutical Composition 40: Any one of Pharmaceutical Compositions 24 through 26, wherein the vasodilator is hydralazine.

Pharmaceutical Composition 41: Any one of Pharmaceutical Compositions 24 through 26, wherein the vasodilator is an angiotensin receptor-neprilysin inhibitor.

Pharmaceutical Composition 32: Pharmaceutical Composition 41, wherein the angiotensin receptor-neprilysin inhibitor is sacubitril/valsartan.

Method 43: A method for the promotion of neo capillary formation in a patient in need thereof, comprising: administering an effective amount of the pharmaceutical composition according to any one of Pharmaceutical Compositions 24 through 43 to a patient in need thereof, whereby neo capillaries are formed.

Any of the features the above referenced pharmaceutical compositions, uses, and methods is applicable to any other pharmaceutical composition, use, or method identified herein. Moreover, any of the features of the above referenced pharmaceutical compositions, uses, and methods is independently combinable, partly or wholly, with other embodiments of the pharmaceutical compositions, uses, and methods described herein in any way, e.g., one, two, or three or more features may be combinable in whole or in part. Further, any of the features of the pharmaceutical compositions, uses, and methods described above may be made optional to other pharmaceutical compositions, uses, and methods described herein. Any aspect or embodiment of a method or use described herein can be performed using a composition, e.g., a pharmaceutical composition and/or a compound of Formula (I) as described herein or any compound having a structure described herein, and any aspect or embodiment of a composition, e.g., a pharmaceutical composition and/or a compound of Formula (I) or any compound having a structure described herein, can be used or adapted to perform a method or use as described herein.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method for the treatment of fibromyalgia in a patient in need thereof, comprising:
topically administering an effective amount of a composition comprising at least one vasodilator to a patient in need thereof,
thereby treating a symptom of fibromyalgia.

2. The method of claim 1, wherein the symptom is pain, myofascial pain, or soft tissue pain.

3. The method of claim 1, wherein the vasodilator is a calcium channel blocker.

4. The method of claim 3, wherein the at least one calcium channel blocker is a dihydropyridine selected from the group consisting of nifedipine, isradipine, felodipine, amlodipine, nicardipine, and clevidipine.

5. The method of claim 1, wherein the composition is in a form adapted for topical administration to an epidermis of the patient in need thereof.

6. The method of claim 5, wherein the form is selected from the group consisting of a cream, a lotion, an ointment, a gel base, a foam, a powder, an aerosol spray, an oil, a liquid, and a suspension.

7. The method of claim 1, wherein the composition is administered once a month.

8. The method of claim 1, wherein the composition further comprises a carrier selected from cyclosiloxane or linear silicone.

9. The method of claim 8, wherein the carrier comprises 80 wt % to 95 wt % of the composition.

10. The method of claim 1, wherein the composition further comprises one or more of a diluent, an excipient, a wetting or emulsifying agent, a pH buffering agent, a gelling or viscosity enhancing additive, a preservative, a scenting agent, or a coloring agent.

11. A method for the promotion of neo capillary formation, the comprising:
topically administering an effective amount of a composition comprising at least one vasodilator to a patient in need thereof,
thereby promoting neo capillary formation.

12. The method of claim 11, wherein the vasodilator is in a form adapted for topical administration to an epidermis.

13. The method of claim 12, wherein the form is selected from the group consisting of a cream, a lotion, an ointment, a gel base, a foam, a powder, an aerosol spray, an oil, a liquid, and a suspension.

14. The method of claim 11, wherein the vasodilator is a calcium channel blocker.

15. The method of claim 14, wherein the at least one calcium channel blocker is a dihydropyridine selected from the group consisting of nifedipine, isradipine, felodipine, amlodipine, nicardipine, and clevidipine.

16. The method of claim 11, wherein the composition is administered once a week.

17. The method of claim 11, wherein the composition is administered once a month.

18. The method of claim 11, wherein the composition further comprises a carrier selected from cyclosiloxane or linear silicone.

19. The method of claim 18, wherein the carrier comprises 80 wt % to 95 wt % of the composition.

20. The method of claim 11, wherein the composition further comprises one or more of a diluent, an excipient, a wetting or emulsifying agent, a pH buffering agent, a gelling or viscosity enhancing additive, a preservative, a scenting agent, or a coloring agent.
